(19) United States
(12) Patent Application Publication
(10) Pub. No.: US 2019/0255367 A1
(43) Pub. Date: Aug. 22, 2019

(54) PERSONALIZED FORCED AIR PURIFIER

(71) Applicant: GHOLAM HOSSEIN ZERESHKIAN, Richmond Hill (CA)

(72) Inventor: GHOLAM HOSSEIN ZERESHKIAN, Richmond Hill (CA)

(21) Appl. No.: 16/400,197

(22) Filed: May 1, 2019

Related U.S. Application Data
(63) Continuation-in-part of application No. 14/966,494, filed on Dec. 11, 2015.

(51) Int. Cl. A62B 7/10; A62B 23/02; A62B 18/00; B01D 46/00; B01D 47/02; B01D 50/00; C02F 1/46; C02F 1/461; B03C 1/28; A61L 9/22

(52) U.S. Cl. CPC A62B 7/10; A61L 2209/14; A62B 18/003; A62B 18/006; B01D 46/0035; B01D 46/0028; B01D 47/024; B01D 50/006; C02F 1/4606; C02F 1/46109; B03C 1/28; A61L 9/22; C02F 2001/46133; C02F 2303/04; B03C 2201/20; B01D 2275/403; A61L 2209/134; A62B 23/02

(57) ABSTRACT

The present invention is a portable air purifier device intended to be attached to the belt or to the neck of a user. Purified air is generated by electrochemically and magnetically treating a polluted air. Polluted air is forced through an ionized water, which absorbs particles and kills possible air born bacteria. A magnetic filter assures that small metal particles are also separated from the polluted air. The purified air is then delivered towards the face of the user either through an air curtain or through a mask.

### CROSS REFERENCE TO RELATED APPLICATIONS

This application is a continuation-in-part of U.S. patent application Ser. No. 14/966,494, which was filed on Dec. 11, 2015. The applications are herein incorporated by reference for all that they contain.

### FIELD OF THE INVENTION

The present invention relates in general to the field of air purifiers, and in particular to a personalized forced air purifier system and mask.

### BACKGROUND OF THE INVENTION

A wide variety of face masks have been developed to protect people from polluted particulate matter and dust. Most of the dust masks have been developed to protect people from larger particulate matters such as sand, wood dust, metal dust or the like. Many filtering masks are designed for removing particles as well as some gasses that may include bacteria or harmful viruses. Filtered masks in the market are not efficient and need high intake pressure, which makes using them difficult, especially for the elderly. These masks are not convenient and they usually cover most of the face. Additionally, they tend to be cumbersome and expensive to manufacture and purchase.

The prior art masks that are mainly used to filter dust and particles, may not specifically filter small metal particles. Small particles of metals such as iron that come from fuel additives, brakes and tires attrition enter into the respiratory system and reach the systemic circulation. In the blood, metals are attached to proteins or ionized entering into the different organs and cells producing variety of outcomes.

Increased risk caused by metal particles result to serious health issues, including increased inflammatory responses in the brain, producing DNA damages, low birth-weight, infertility, genotoxicity and cancer such as leukemia and lymphoma. Also, metals may interact directly with the components of the cell leading to cell death.

Environmental pollution by metal particles is a public health problem, which also affects the central nervous system. the scientists have found a relationship between Alzheimer Disease and very fine metal particles in polluted air, which pass through lung system to brain and cause the disease. These health issues have resulted in behavior, learning, mental disorders, attention deficit and low mental performance especially cognitive ability in children resulting in an IQ decrease. The present device removes small metal particles from an air flow.

### SUMMARY OF THE INVENTION

The present invention is an air purifier in combination with a mask or an air curtain to provide clean breathing air. The air purifier comprises of a small container, which can be attached to a belt or hang from the neck of a user. The container is preferably made of a light weight material and includes a top lid, a bottom wall and side walls adapted to house a water absorbing material of any one of sponge, cotton etc., which can be removed to be cleaned. The filter is preferably from a material with strong absorbability and is wetted by water or ionized water. The wet filter has an inlet area and an outlet area. The inlet area of the wet filter is exposed to the ambient air. The container has a plurality of openings on the top lid and plurality of openings on the side walls. The container may have any shape and size. A battery driven air fan is attached to the outlet area of the wet filter to suck the ambient air through the wet filter and exhaust a purified air. In operation, the fan sucks the ambient air passing through the filter in the container and exhausts a clear flow of air.

In another embodiment of the present invention the purifier has an ionization system embedded inside the container, which has a water chamber. The ionization system comprises of two plate electrodes to ionize and disinfect the water inside the container. The electrodes are from a heavy metal, such as a copper-silver alloy. Each electrode is connected to a power source. The ions released in the wet filter kill the bacteria released from the ambient air through electrochemical process.

The present air purifier also has permanent magnets to capture the metal particles in the air.

When a person breath in, the contaminated air is driven into the container and pollution, dust and contaminants of the air are scrubbed by the filter and neutralized by the ionized water. The purified air is then released into the container-outlet-port. A flexible air hose is connected to the outlet-port to carry the purified air into the flexible air hose and guide it to the user's interface.

Fastening means are provided on the side walls of the container to open the top lid of the container and replace the contaminated filter, which contains dust and dead bacteria, and replace it easily with a new filter. The personal air purifier can be attached to a person's belt using any different type of securing means such as a belt with a belt buckle, or it can be hung on a person's neck.

The output of the container is connected to a mask or an air curtain system, through a flexible air hose. The air curtain system can be mounted on the users chin by a chin-attaching means. The air curtain system comprises of a series of air jets at the outlet a connector that connects to the outlet of the air hose. Flexible hose guides the purified air to the users face and generates a curtain of purified air in front of the nose and mouth of the user.

The air purifier of the present invention can also be used with a full face cover mask having an opening in front of it to direct the exhale air out of the mask. The mask is held onto the user's head by standard elastic straps.

The container further comprises of a power source to provide power to the fan. The power source is a DC battery or other suitable batteries that can be used as a power source.

The present invention can be used in hospitals to protect patience against pandemic air born disease. It can also be used in dusty areas to filter the air, which is essential especially for people with allergy or asthma.

The present invention can also be equipped with a container for medical solutions to treat a variety of diseases. Inhalation solutions are typically aqueous-based formulations and contain therapeutically active ingredients and can be used by the present invention.

The present invention can also be equipped with a container for applying personalized aromatic fluids and good smells for the particular use of the user in areas with undesirable smells.

It is an object of the present invention to provide an air purifying mask, which is light, non-expensive, easy to use and safe.

It is another object of the present invention to provide such a device which is highly portable, and which would be lightweight and inexpensive to construct.

Other objects, features, and advantages of the present invention will be readily appreciated from the following description. The description makes reference to the accompanying drawings, which are provided for illustration of the preferred embodiment. However, such embodiments do not represent the full scope of the invention.

### BRIEF DESCRIPTION OF THE DRAWINGS

Embodiments herein will hereinafter be described in conjunction with the appended drawings provided to illustrate and not to limit the scope of the claims, wherein like designations denote like elements, and in which:

FIG. 1 is a front view of the personalized forced air purifier according to the present invention;

FIG. 2 is a side view of personalized forced air purifier of the present invention and the filter;

FIG. 3 is a top view of the personalized forced air purifier in open position according to the present invention;

FIG. 4 is a top view of another embodiment of the present invention;

FIG. 5 is a top view of another embodiment of the present invention;

FIG. 6 is a perspective view of a chin-attaching mask according to the present invention;

FIG. 7 is a perspective view of another type of mask portion of the present invention;

FIG. 8 is a perspective view of another embodiment of the personalized forced air purifier of the present invention;

FIG. 9 is a perspective view of another embodiment of the present invention with magnetic purifying system;

FIG. 10 is a perspective view of another embodiment of the magnetic purifying system;

FIG. 11 is a perspective view of another embodiment of the magnetic purifying system, and

FIG. 12 is a perspective view of another embodiment of the magnetic purifying system.

### DETAILED DESCRIPTION OF PREFERRED EMBODIMENTS

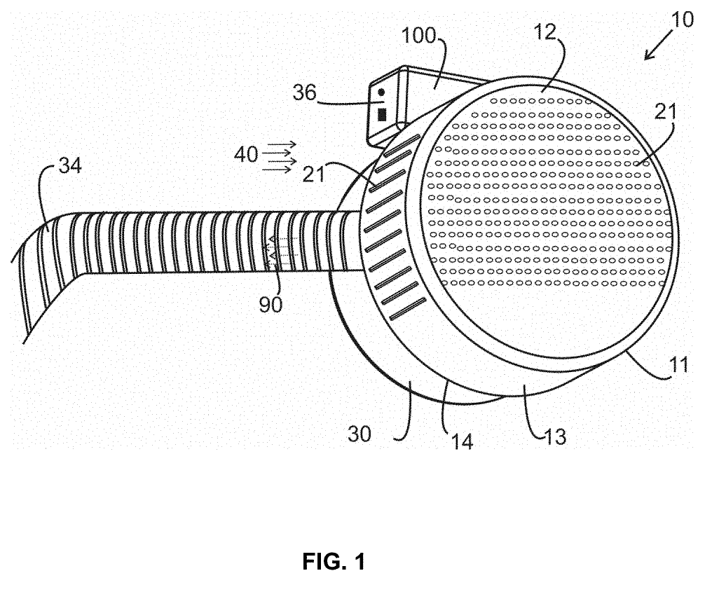

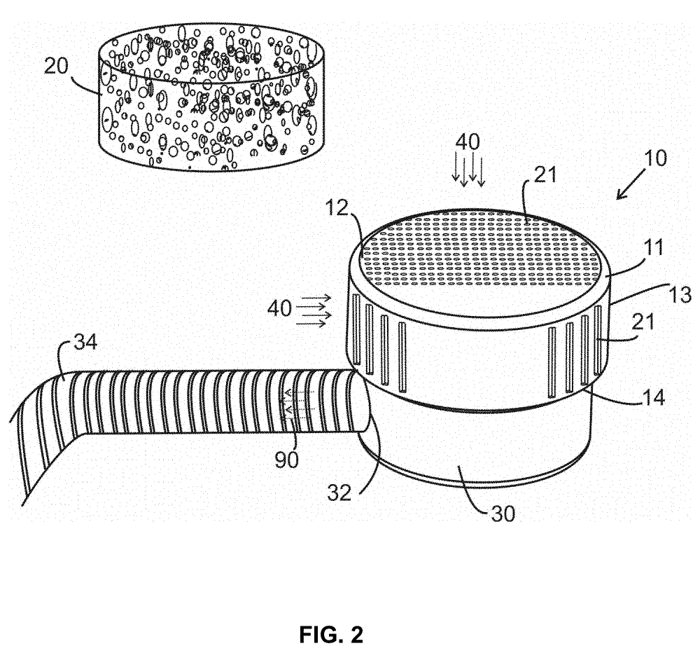

Referring to FIGS. 1 and 2, the present personalized forced air purifier is a portable wet filter 10 comprising of a container 11 preferably made of a light weight material. The container 11 includes a top lid 12 and a bottom wall 14 and side walls 13. Said container is adapted to house a filter. The top lid 12 has perforations 21 to allow the ambient air 40 to enter into the container for purifying. The lid 12 is fastened on top of the container 11 by a fastening means. There are also openings 21 provided on the side walls 13 of the container 11. The container 11 is adapted to house a sponge like filter. The filter 20 is sponge like material with strong absorbability and is wetted by water or ionized water. The filter 20 can be replaced after contaminated with a clean filter.

A battery driven air fan 30 is attached to the bottom wall 14 of the container 11. The air fan 30 sucks the ambient air 40 through the filter and exhausts a purified air 90 and directs it through an outlet-port to the flexible hose 34. The flexible hose 34 is connected to the outlet-port from one end and to a mask from another end.

A power source 100 is provided on the side wall 13 of the container to provide power to the fan 30. The power source is a DC battery or other suitable batteries. Li-Ion battery driven fans are readily available, which can provide sufficient air flow.

The air purifier 10 is equipped with a control panel 36 to manage the speed of the air fan 30 and the operation of the power source 100.

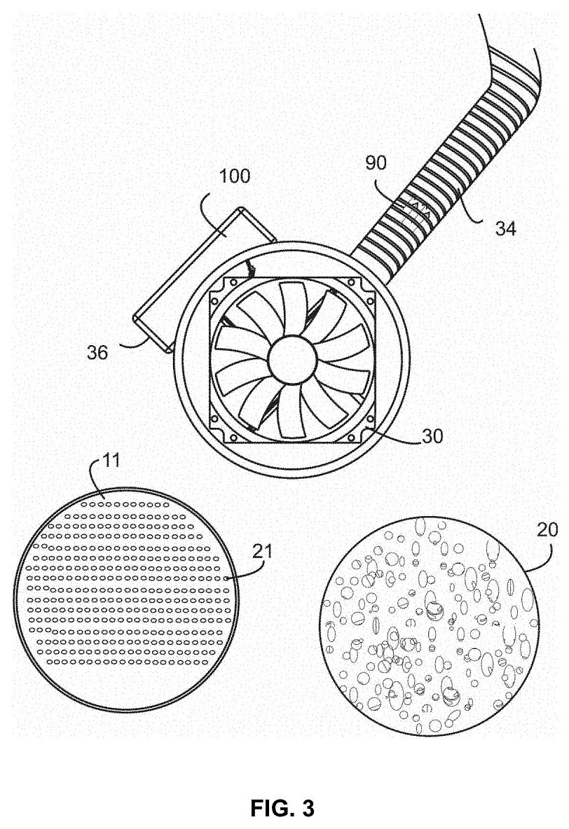

FIG. 3 is a top elevation of the inner part of the personalized forced air purifier 10. The air fan 30 is mounted on the bottom wall 14 of the container and sucks the contaminated air through the openings 21 into the container 11. The filter 20 is inserted into the container and stands in a higher elevation from the fan 30. The contaminated air passes through the filter 20 and the pollution, dust and contaminants of the air are scrubbed by the filter 20. The purified air 90 is then diverted into the air hose 34.

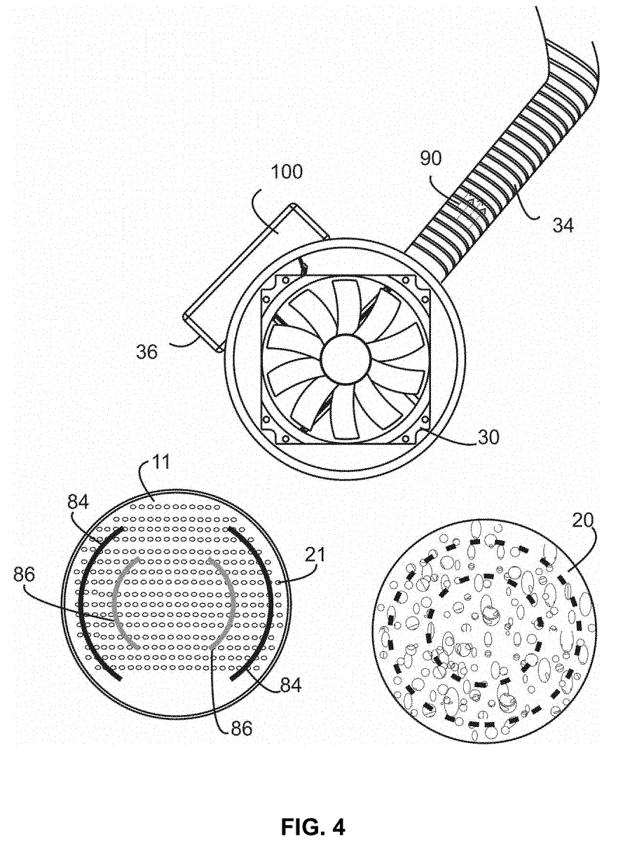

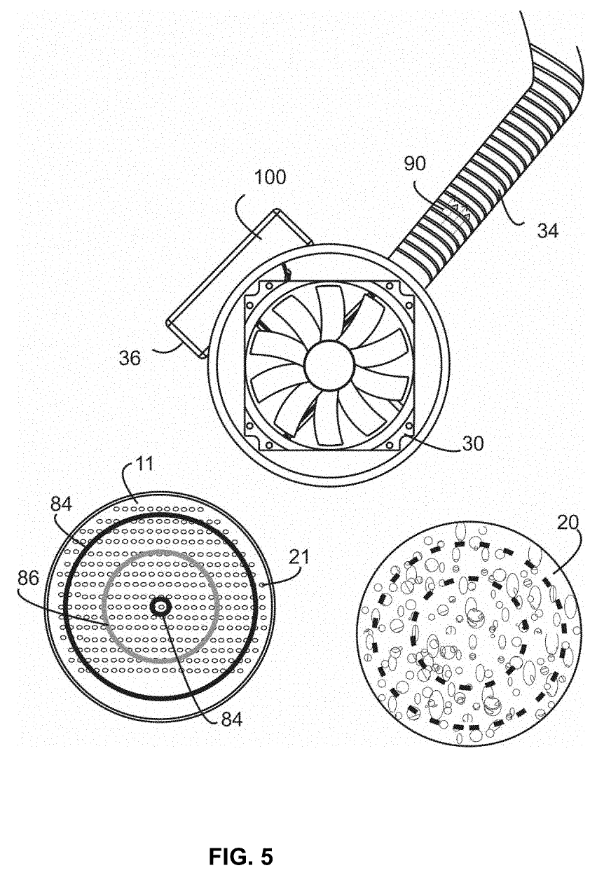

According to FIGS. 4 and 5 in another embodiment of the air purifier 10 the filtering system comprises a combination of wet filter 20 and ionization system. An ionization system is embedded inside the container 11. The ionization system comprises of a series of electrodes to produce ions and disinfect the air inside the container 11. A variety of electrode configurations can be used for purification. FIG. 4 shows two curved plate electrodes 84-86. The electrodes are from a heavy metal, such as a copper-silver alloy. Each electrode 84-86 is being connected to the power source 100. Released ions act as an electrochemical treatment to kill the bacteria released from the ambient air 40. The electrodes 84-86 can be in various configurations and the wet filter 20 is then embedded in between the electrodes.

The fan 30 sucks a significant amount of ambient air into the container 11 and pollution, dust and contaminants of the air are scrubbed by the filter 20 and neutralized by the ions. The purified air 90 is then released into the flexible hose 34 through a container-outlet-port. The purified air is then carried towards the user's face.

Referring to FIGS. 1, 2, 3 and 4 a flexible air hose 34 is connected to the outlet port 32 of the container. The air hose 34 receives the purified air 90 and directs it to the user's face. The flexible hose 34 is connected to the outlet-port 32 from one end and to a mask or another air distribution system from another end.

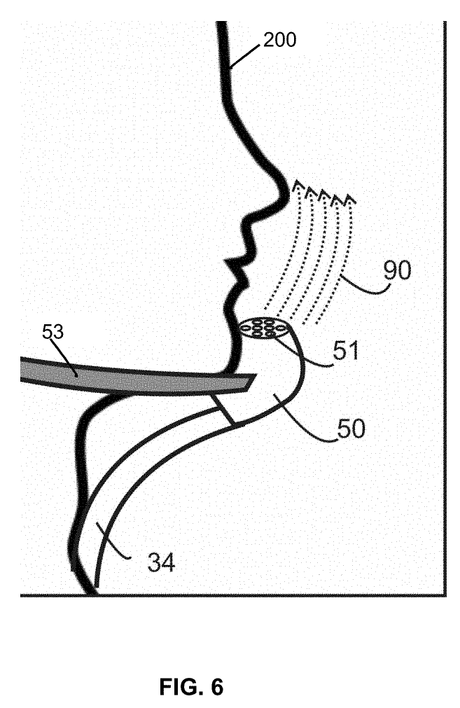

The personalized forced air purifier 10 of the present invention is equipped with means to generate a flow of purified air in front of user's face. As shown in FIG. 6 said means can be a set of air jets 51 provided on a chin attaching air curtain 50. Said air curtain 50 can be attached on the chin of a user 200. Plurality of air nozzles 51 are provided on the proximal end of the flexible hose 34 and extended to the front portion of the air curtain 50. The air jets on the air curtain generate a curtain of purified air 90 in front of the nose and mouth of the user. The air curtain 50 is attached to the user's chin using an elastic strap 53 or any other strapping means.

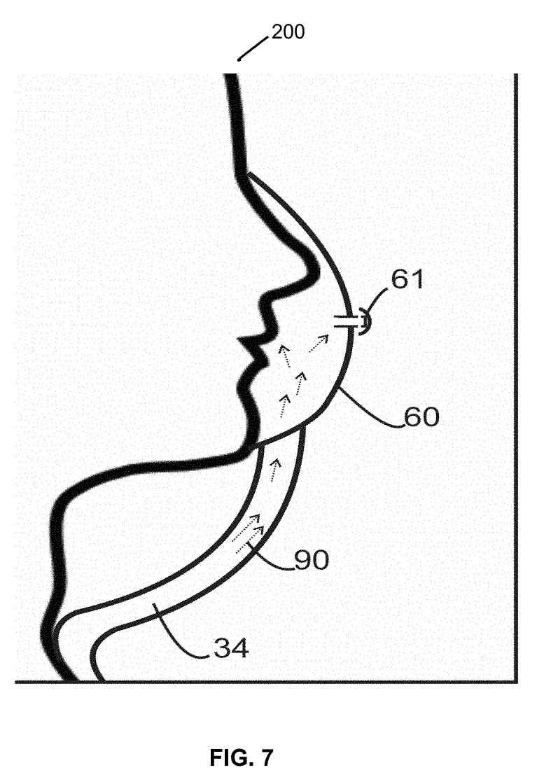

Referring to FIG. 7, air generating means can be a face mask 60 having an opening 61 in front of it. This enables the user 200 to inhale and exhale the forced purified air through the mask 60. Because the purified air is forced through the mask and there is a continuous flow of purified air in and out of the mask, the unpurified air cannot enter the mask. The purified air 90 is directed into the mask 60 through the flexible hose 34 and the exhaled air is carried out of the mask 60 through the opening 61.

The personal air purifier 10 is made of a light weight material to be attached to a belt or hang from a neck of a user. According to FIGS. 6 and 7, the flexible hose 34 and Interface means 50-60 may comprise of rigid or resilient material or clear plastic material. Adjustable straps 53 can be used to attach the mask to the persons chin or head to allow the nose and mouth breathe the purified air 90.

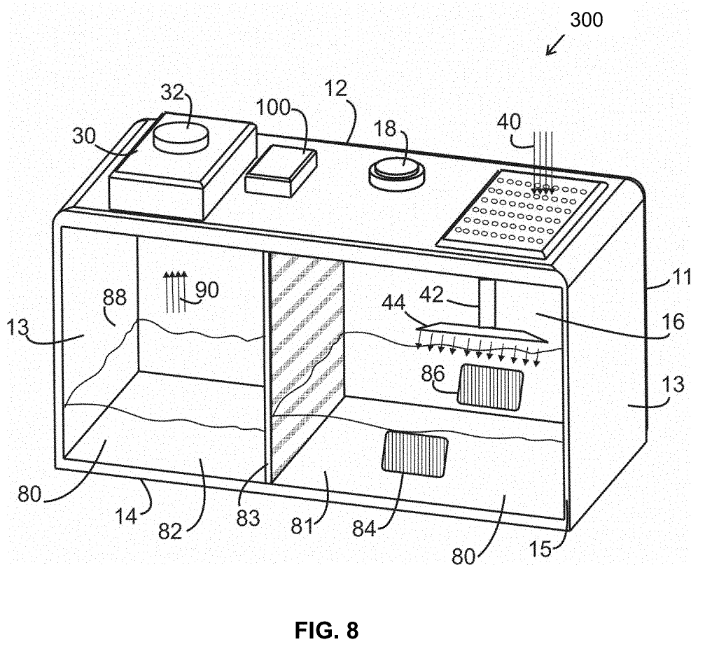

FIG. 8 shows another embodiment of the present invention. The personal air purifier 300 comprises a container 11 having a top wall 12, a bottom wall 14, a front wall 15 and a rear wall 16 and side walls 13. Said container is divided to two sections by a filter 83 and adapted to house a volume of ionized water 80. An ionization system is embedded inside the water section 80 of the container 11. It is well known that ions of heavy metals such as copper and silver are biocidal for many bacteria. The ionization system comprises of two plate electrodes 84-86 attached to the inner walls of said compartment to ionize and disinfect the water inside the container 11. The first electrode 84 is attached to the front wall 15 of the container and the second electrode 86 is attached to the rear wall 16 of the container. The electrodes 84-86 deposit specific amount of ions into the water 80. Each electrode 84-86 is connected to the power source 100. Released ions in the water act as an electrochemical treatment to kill the bacteria released from the ambient air 40 into the water.

A battery driven air fan 30 is attached to the top wall 12 of the container. The air fan 30 sucks the ambient air 40 through openings into the container 11. The ambient air directs into the container through an air conduit 42. The air conduit 42 extends to the top of the water surface 88. A variety of air nozzles 44 can be attached to the exit of the air conduit 42 to change the air velocity impinging on the water surface 88.

Referring again to FIG. 8 again a filter 83 divides the container 11 into two compartments. The first compartment 81 is on one side of the filter 83, on the side of the air conduit 42, where the contaminated air 40 is injected. The second compartment 82 is on the other side of the filter 83, in which the air is purified. The pollution, dust and contaminants of the air are scrubbed by the water and neutralized by the ionized water. The purified air 90 entering into the clean compartment 82 is guided towards the users face.

The air fan 30 is strong enough to generate 10-15 lit/min output flow of breathing air. When this high flow air impinges on the water at the bottom of the container, it generates a significant amount of mixing and turbulence. The splashing water droplets scrub the contaminants, dust and other pollutants from the air. Any other particle that is not absorbed by the water is filtered through the filter 83. The fan 30 is battery operated to make it portable. Any type of battery driven fan can be used. Li-Ion battery driven fans are readily available which can provide sufficient air flow.

A power source 100 is provided on top wall 12 to provide power to the fan and the ionization system. Said power source is a DC battery or other suitable batteries.

The purified air 90 is then released in the second compartment 82. An outlet-port 32 is provided on top wall 12 of the second compartment 82. The flexible hose is then connected to the outlet-port 32 to carry the purified air 90 into the flexible hose and guide it to the user's interface.

As shown in FIG. 8 again an aperture 18 is provided on the top wall 12 to fill the container 11. In operation, one need only pour a measured amount of water through the aperture 18. All of the openings are positioned so as to prevent any liquid water from leaking out of the container 11. Purified air 90 is provided towards the users' face through hose, allowing for the user to breathe clean air. Ionized water which contains dust and dead bacteria will be replaced/refilled easily through the same aperture 18.

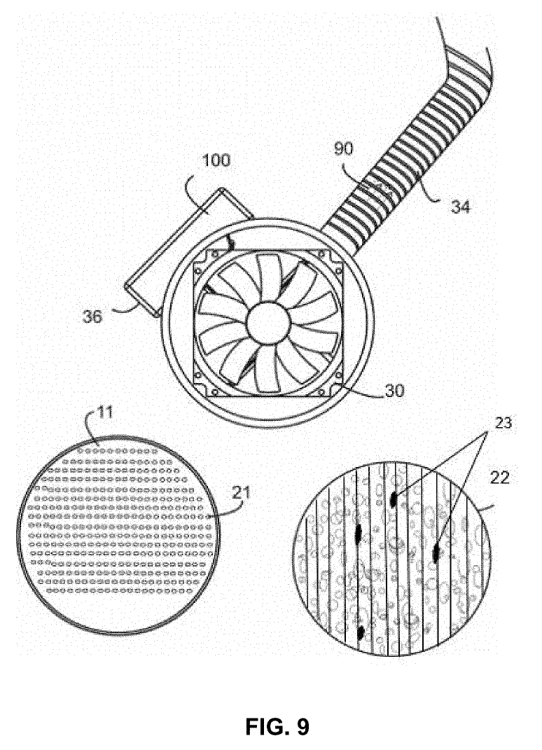

In another embodiment the air purifying device of the present invention 10 has magnetic filter to purify the inhaling air from toxic metal particles. Among these metal particles, Iron (Fe), nickel (Ni) and Cobalt (Co) are extremely dangerous and toxic to human health. The magnetic filters work by producing a magnetic field or loading zones that collect nanoparticles of metals. Magnets are geometrically arranged to form a magnetic field. The magnetic filters can be provided in various configuration. In one configuration, as shown in FIG. 9, the magnetic filter 22 is a mesh shape magnetic plate 22, which can be embedded under the top lid 21 of the container or in an elevation from the top lid 21. The magnetic plate 22 is a circular plate with a diameter equal to the top lid 21 and have attaching means to be attached to the peripheral edges of the top lid 21. The magnetic filter may also magnetically self-cling to the inside of the container 11. Other embodiments of the invention feature different optional magnetic filter profile configurations.

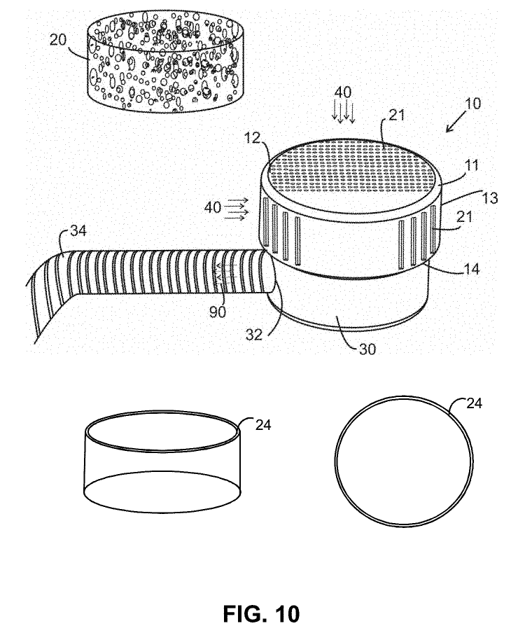

Referring to FIG. 10, in another configuration, a magnetic mesh ring 24 is placed in the container perpendicularly to the side wall 13. This mesh ring 24 can have a diameter equal to the side wall 13 of the container or smaller than the side wall 13 and have attaching means to be attached to the peripheral edge of the side wall 13 or magnetically self-cling to the inside of the side wall 13.

In another embodiment of the present invention, the top lid 12 is made of magnet, which can be acted as a magnetic filter.

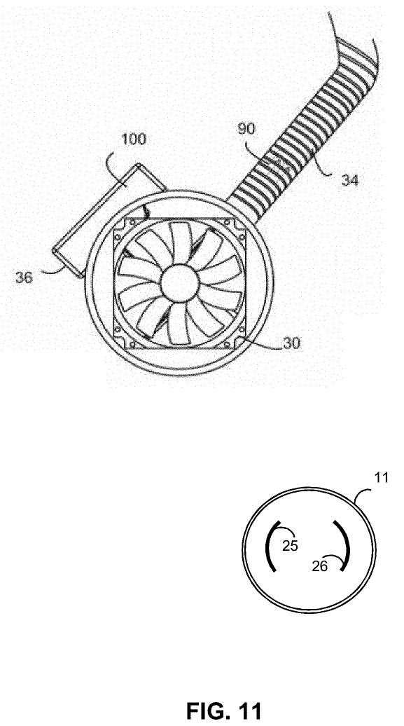

According to FIG. 11, in another embodiment, the magnetic filter comprises of two permanent magnets 25-26 embedded inside the container 11 to produce a strong magnetic field and attract the metal particles of the ambient air 40 entering into the container 11. Each magnet 25-26 has respectively an elongated circular or rectangular-shaped profile. The magnetic filters 25-26 self-cling magnetically to the inside of the container.

In each configuration, the magnetic filter generates a magnetic field which attracts the metal particles 23 from ambient air 40 and acts as a magnetic treatment. In all configuration the wet filter 20 is then embedded in between the magnets. All configuration of the magnetic filter is placed in the container 11 in combination of other parts of the air purifier 10 to eliminate any possibility to cause any reduction of the air flow for passing through the container 11.

The contaminated air 40 passes through the filter 20 and the pollution, dust and contaminants of the air are scrubbed by the filter 20. The ionization system produces ions and disinfect the air inside the container and the magnet filter absorb the metal particles 23 of the air. The purified air 90 is then diverted into the air hose 34. The arrangement of the electrodes 84-86 and magnets are so that the ambient air can receive an electrochemical and magnetically treatment. The purified air 90 is then released into the flexible hose 34 through a container-outlet-port 32. The purified air is then carried towards the user's face.

The magnetic filter can be easily installed and replaced without requiring any form of skills or tools. Various types of magnets can be used in these filters. The magnetic filters are made of material selected from the group, but not limited to, ceramic magnets or neodymium iron boron magnet. Periodically the magnets are removed and wiped clean.

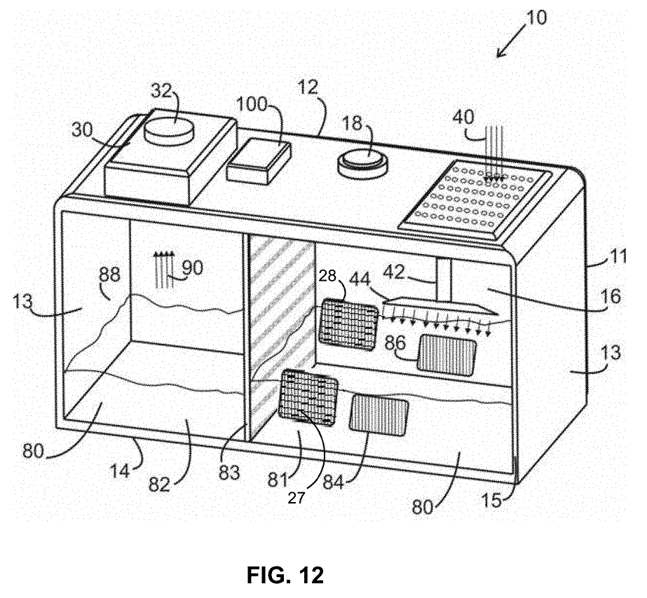

According to FIG. 12, another embodiment of the magnetic purifier is disclosed. The magnet purifier comprises of two electromagnets 27-28 electrically connected to the power source of the purifier device 10. The electromagnets generate a magnetic field to attract the metal particles. The electromagnets 27-28 are attached to the inner walls of said compartment 81. The first electromagnet 27 is attached to the front wall 15 and the second electromagnet 28 is attached to the rare wall 16 of the compartment 81. Each electromagnet 27-28 is connected to the power source 100 and generate a magnetic field inside the compartment 81.

The contaminated air 40 is injected into the compartment 81 and purified by ionization system and magnetic system. The purified air 90 entering into the clean compartment 82 is guided towards the users face. Any other particle that is not absorbed by the water and magnets is filtered through the filter 83. The purified air 90 is then released in the second compartment 82. An outlet-port 32 is provided on top wall 12 of the second compartment 82. The flexible hose is then connected to the outlet-port 32 to carry the purified air 90 into the flexible hose and guide it to the user's interface.

The foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

With respect to the above description, it is to be realized that the optimum relationships for the parts of the invention in regard to size, shape, form, materials, function and manner of operation, assembly and use are deemed readily apparent and obvious to those skilled in the art, and all equivalent relationships to those illustrated in the drawings and described in the specification are intended to be encompassed by the present invention.

What is claimed is:

1) A personalized forced air purifier comprising:
a) a container having an inlet area and an outlet area, wherein the inlet area of the container is exposed to an ambient air;
b) a wet filter to absorb and retain a mass of water, the wet filter placed inside the container;
c) a set of ionization electrodes embedded inside the container to generate ions inside the water;
d) a magnet embedded inside the container to generate a magnetic field;
e) an air fan attached to the outlet area to suck the ambient air through the wet filter and to exhaust a purified air;
f) a power supply to power the air fan and the set of ionization electrodes; and
g) a face mask to receive the purified air from the fan through an air hose, the face mask having an outlet port to let the forced air to pass through the mask, thereby allowing a user of the face mask to breath the purified air,
whereby the ionization electrodes and the magnet provide an electrochemical treatment and a magnetic treatment of the air inside the container.

2) The personalized air purifier of claim 1, wherein the container comprising of a lid, a bottom surface, and side walls, the lid having perforations to allow air to enter the container, and the set of ionization electrodes being attached to the side walls.

3) The personalized air purifier of claim 2, wherein the set of ionization electrodes are attached to the bottom surface and extend into the container, and the wet filter is located in between the ionization electrodes.

4) The personalized air purifier of claim 2, wherein the magnet plates are attached to the top lid.

5) The personalized air purifier of claim 2, wherein the magnet ring is attached to the side walls.

6) The personalized air purifier of claim 1, wherein the wet filter is a sponge or a cotton.

7) The personalized air purifier of claim 1, wherein the magnet comprising of a set of magnet plates or magnet rings.

8) The personalized air purifier of claim 1, wherein the magnet comprising of a plurality of magnet plates or magnet rings embedded inside the container and the wet filter is fitted in between the magnet plates or magnet rings.

9) The personalized air purifier of claim 1, wherein the magnet is a neodymium iron boron magnet, a sintered magnet, a nickel-cobalt material, or an electromagnet.

10) The personalized air purifier of claim 1, wherein the ionization electrodes are copper or silver electrodes.

11) A personalized air purifier comprising:
a) a container to be partially filled with water and to have a water surface, the container having a first and a second compartments;
b) a filter fitted in between the first and the second compartments;
c) an air fan attached to a top surface of the container;
d) an air conduit inside the first compartment having an inlet and an outlet, wherein the inlet is attached to an outlet-port of the fan, the conduit directing air towards the water surface, and the outlet generating a high velocity air flow impinging on the water surface to generate a highly turbulent water and water droplets, whereby the air is purified by the water droplets;
e) a pair of electrodes attached to the inner walls of the first compartment to ionize and disinfect the water inside the container, the second compartment to receive a purified air, the purified air being the air that moves from the first compartment through the filter to the second compartment;
f) a pair of magnets attached to the inner walls of the first compartment to attract metal particles inside the container, the second compartment to receive a purified air, the purified air being the air that moves from the first compartment through the filter to the second compartment;
g) a power source to provide power for the air fan and the pair of electrodes and the pair of magnet plates;
h) a control panel to manage a speed of the air fan and to operate the power source;
i) a container-outlet-port at the top of the second compartment; and
j) a flexible pipe attached from one end to the container-outlet-port and to carry the purified air towards a user's face.

12) The personalized air purifier of claim 11, wherein the ionization electrodes are copper or silver electrodes.

13) The personalized air purifier of claim 11, wherein the power source is a DC battery.

14) The personalized air purifier of claim 11, wherein the air conduit extends to the top of the water surface, and wherein a nozzle is attached to the outlet of the air conduit to increase the air velocity impinging on the water surface, thereby generating turbulence and rapid mixing between the air and the water.

15) The personalized air purifier of claim 11, wherein the fan being battery operated to provide an air flow.

16) The personalized air purifier of claim 11, wherein the container further having an aperture on the top wall to fill and refill the container.

17) The personalized air purifier of claim 11, wherein the flexible pipe having a nozzle to generate a curtain of purified air in front of the nose and the mouth of the user.
